(19) United States
(12) Patent Application Publication
(10) Pub. No.: US 2010/0119824 A1
(43) Pub. Date: May 13, 2010

(54) PHENOLIC RESIN, METHOD OF PREPARATION, SIZING COMPOSITION FOR MINERAL FIBRES, AND RESULTING PRODUCTS

(75) Inventors: Vincent Gignoux, Senlis (FR); Serge Tetart, Saint-maximin (FR); Olivier Pons Y Moll, Agnetz (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: SAINT-GOBAIN ISOVER, Courbevoie (FR)

(21) Appl. No.: 12/445,363

(22) PCT Filed: Oct. 11, 2007
(86) PCT No.: PCT/FR07/52121

§ 371 Date: May 13, 2009

(30) Foreign Application Priority Data
Oct. 11, 2006 (FR) 0654200

(51) Int. Cl. D06M 15/41; C07C 223/00; B32B 27/04

(52) U.S. Cl. 428/392; 564/502; 252/8.83

(57) ABSTRACT

The present invention relates to a liquid phenolic resin, intended to be used in the formulation of a sizing composition for mineral fibres, essentially consisting of phenol-formaldehyde and phenol-formaldehyde-amine condensates and having a water dilutability at 20° C. of at least 1000%, and it has a free formaldehyde content of 0.3% or less and a free phenol content of 0.5% or less, the contents being expressed with respect to the total weight of liquid.

The subject of the invention is also a method of manufacturing said resin, the sizing composition containing the resin and the insulating products based on mineral fibres sized by means of the aforementioned sizing composition.

The invention relates to a phenolic resin intended to be used in the formulation of a sizing composition for mineral fibres. This resin is obtained by the condensation of phenol, formaldehyde and an amine in the presence of a basic catalyst, and it is characterized by a low content of free formaldehyde and free phenol.

The invention relates to a method of preparing this resin, to the sizing composition for mineral fibres that contains said resin, and to the insulating products that result therefrom.

Insulation products based on mineral fibres may be formed from fibres obtained by various processes, for example using the known technique of internal or external centrifugal fiberizing. The centrifugation consists in introducing molten material (in general glass or rock) into a spinner that has a multitude of small holes, the material being projected against the peripheral wall of the device under the action of the centrifugal force and escaping therefrom in the form of filaments. On leaving the spinner, the filaments are attenuated and entrained by a high-velocity high-temperature gas stream to a receiving member in order to form a web of fibres.

To assemble the fibres together and provide the web with cohesion, the fibres, on leaving the spinner, are sprayed with a sizing composition containing a thermosetting resin. The web of fibres coated with the size undergoes a heat treatment (at a temperature above 100° C.) so as to polycondense the resin and thus obtain a thermal and/or acoustic insulation product having specific properties, especially dimensional stability, tensile strength, thickness recovery after compression, and uniform colour.

The sizing composition is made up of the resin, which in general takes the form of an aqueous solution, of additives, such as urea, silanes, mineral oils, aqueous ammonia and ammonium sulphate, and of water. The sizing composition is usually sprayed onto the fibres.

The properties of the sizing composition depend largely on the characteristics of the resin. From the standpoint of the application, it is necessary for the sizing composition to have good sprayability and be able to be deposited on the surface of the fibres so as to bond them effectively. The sprayability is directly related to the capability that the resin possesses of being able to be diluted in a large amount of water and to remain stable over time.

The dilution capability is characterized by the “dilutability”, which is defined as the volume of deionized water that it is possible, at a given temperature, to add to a unit volume of the aqueous resin solution before the appearance of permanent cloudiness. In general, a resin is considered to be able to be used as a size when its dilutability at 20° C. is 1000% or higher.

The resin must still be stable over a given lapse of time before being used to form the sizing composition, which composition is generally prepared at the moment of use by mixing the resin and the abovementioned additives. In particular, the resin must be stable for at least 8 days at a temperature of around 12 to 18° C.

The resin that can be used in a sprayable sizing composition must have dilutability at 20° C. of 1000% or higher, preferably 2000% or higher (infinite dilutability), for at least 8 days.

From the regulatory standpoint, it is necessary for the resin to be considered as non-polluting, that is to say for it to contain—and generate during the sizing operation or subsequently—as few as possible compounds considered to be harmful to human health or to the environment.

The thermosetting resins most commonly used are phenolic resins belonging to the family of resoles. Apart from their good crosslinkability under the aforementioned thermal conditions, these resins are very soluble in water, possess good affinity for mineral fibres, especially glass fibres, and are relatively inexpensive.

These resins are obtained by the condensation of phenol and formaldehyde, in the presence of a basic catalyst, in a formaldehyde/phenol molar ratio generally greater than 1 so as to promote the reaction between the phenol and the formaldehyde and to reduce the residual phenol content in the resin. The residual amount of formaldehyde and phenol in the resin remains high.

To reduce the amount of residual formaldehyde, it is known to add a sufficient amount of urea to the resin, the urea reacting with the free formaldehyde, forming urea-formaldehyde condensates (see EP 0 148 050 A1). The resin obtained contains phenol-formaldehyde and urea-formaldehyde condensates, has a free formaldehyde and free phenol content, expressed with respect to the total weight of liquid, of 3% and 0.5%, respectively, or less, and a water dilutability of at least 1000%.

Although the amount of residual phenol is acceptable, the amount of residual formaldehyde is however too high to meet the current regulatory constraints.

Moreover, it has been found that the resin is not stable under the conditions that are applied during the treatment of the sized fibres for the purpose of crosslinking the resin in order to form the final insulating products. At the temperature of the treatment, generally above 100° C. in an oven, the urea-formaldehyde condensates are degraded and they release formaldehyde, which increases the undesirable gas emissions into the atmosphere. Formaldehyde may also be released from the end product during its use as thermal and/or acoustic insulation.

EP 0 480 778 A1 has proposed to substitute part of the urea with an amine, which reacts with the free phenol and the free formaldehyde via the Mannich reaction to form a condensation product having improved thermal stability. The free phenol and free formaldehyde contents of this resin are 0.20% or less and 3% or less, respectively.

One subject of the present invention is a phenolic resin which has characteristics sufficient for it to be used in a sprayable sizing composition, which has a low capacity for producing undesirable emissions, especially by having a low free formaldehyde content and a low free phenol content, and which generates little formaldehyde during its use.

Another subject of the invention is a method of producing the resin, which does not involve urea in order to reduce the free formaldehyde content.

Another subject of the invention is a sizing composition comprising said resin, its use for sizing mineral fibres, with the view to forming thermal and/or acoustic insulation products, and the products thus obtained.

The liquid resin according to the invention, intended to be used in a sizing composition for mineral fibres, essentially contains phenol-formaldehyde (P-F) and phenol-formaldehyde-amine (P-F-A) condensates. The resin has a free formaldehyde content of 0.3% or less and a free phenol content of 0.5% or less, these contents being expressed with respect to the total weight of liquid.

Preferably, the resin has a free formaldehyde content of 0.2% or less with respect to the total weight of liquid, and advantageously of 0.1% or less.

Preferably, the free phenol content of the resin is 0.4% or less.

The resin has a dilutability, measured at 20° C., of at least 1000%.

The resin is also thermally stable, as it is free of urea-formaldehyde (U-F) condensates known for their degradability under the effect of temperature. As for the P-F-A condensates, these are stable under the aforementioned conditions—they generate little formaldehyde for example—in particular during ageing of the final insulating product.

The amine is chosen from primary amines, which can react with an aldehyde, for example formaldehyde, and an organic compound comprising active hydrogen atoms, for example phenol, to form a Mannich base. This amine satisfies the following general formula:

R—NH₂

in which R represents a saturated or unsaturated, linear, branched or cyclic hydrocarbon group containing 1 to 10 carbon atoms, preferably 1 to 6 carbon atoms.

Particularly preferably, the hydrocarbon group R carries at least one hydroxyl radical.

The preferred primary amine is monoethanolamine.

According to the invention, the phenol/formaldehyde condensation reaction is monitored by a reaction that consists in condensing the free phenol and the free formaldehyde with a primary amine.

To obtain the resin as defined above, the invention provides a method that consists in reacting the phenol with the formaldehyde in the presence of a basic catalyst, in a formaldehyde/phenol molar ratio of greater than 1, in cooling the reaction mixture and in introducing into said reaction mixture, during the cooling, a primary amine that reacts with the free formaldehyde and the free phenol via the Mannich reaction. The invention is characterized in that the primary amine is introduced right from the start of the cooling and the reaction mixture is maintained at the introduction temperature for a time that varies from 10 to 120 minutes.

Preferably, the phenol and the formaldehyde are made to react in a formaldehyde/phenol molar ratio of between 2 and 4, and advantageously less than or equal to 3, to a degree of phenol conversion of greater than or equal to 93%, and cooling of the reaction mixture is started. The cooling takes place at a stage in the condensation that corresponds to a resin that can still be diluted with water (dilutability greater than 1000%).

The expression “degree of phenol conversion” is understood to mean the percentage amount of phenol that has participated in reaction condensing with the formaldehyde relative to the starting phenol content.

According to the invention, the primary amine is added progressively during the cooling, since the reaction between phenol and formaldehyde is exothermic, and the temperature at the moment of addition of the amine is maintained over the time mentioned above, while taking measures to ensure that the dilutability of the resin remains at least equal to 1000%.

The primary amine is introduced right from the start of the cooling, at a temperature that may vary from 50 to 65° C., preferably about 60° C.

The phase during which the temperature is maintained allows the primary amine to react with almost all of the formaldehyde present in the reaction mixture, and consequently allows the free formaldehyde content in the final resin to be lowered down to a value of 0.3% or lower, advantageously of 0.1% or lower. By maintaining the mixture at this temperature, it is also possible to lower the free phenol content in the resin to a value of 0.5% or below, this being particularly advantageous when the formaldehyde/phenol molar ratio is less than 3.

The preparation of the resin takes place under a temperature cycle, which comprises three phases: a heating phase; a first temperature hold; and a cooling phase.

In the first phase, the formaldehyde and phenol are made to react in the presence of a basic catalyst, while progressively heating to a temperature between 60 and 75° C., preferably about 70° C. The formaldehyde/phenol molar ratio is greater than 1, preferably varies from 2 to 4 and is advantageously equal to 3 or less.

The catalyst may be chosen from catalysts known to those skilled in the art, for example triethylamine, lime (CaO) and alkali or alkaline-earth metal hydroxides, for example sodium hydroxide, potassium hydroxide, calcium hydroxide or barium hydroxide. Sodium hydroxide is preferred.

The amount of catalyst varies from 2 to 15%, preferably 5 to 9% and advantageously 6 to 8% by weight relative to the initial weight of phenol.

In the second phase, the temperature of the reaction mixture, which is reached after heating the reaction mixture (end of the first phase), is maintained until the degree of phenol conversion is at least 93%.

The third phase is a cooling phase during which the primary amine is introduced into the reaction mixture so as to start the reaction with the residual formaldehyde and the residual phenol, and thus to form the P-F-A condensates.

The addition of the primary amine takes place progressively owing to the exothermic character of the reaction, as indicated above, and may for example be carried out at a rate of from 1 to 5%, preferably 2 to 4%, by weight of the total amount of amine per minute.

The amount of primary amine, in particular monoethanolamine, is added in an amount of 0.2 to 0.7 mol, preferably 0.25 to 0.5 mol, of amine per mole of starting phenol.

The duration of the primary amine addition may vary from 10 to 120 minutes, preferably 20 to 100 minutes and advantageously 25 to 50 minutes.

Preferably, the addition of the primary amine is carried out at a temperature between 50 and 65° C. and advantageously about 60° C.

After the primary amine has been added, there is a temperature hold by keeping the temperature at the end of introduction for 10 to 120 minutes, preferably at least 15 minutes, so as to continue the condensation reaction between the formaldehyde, the phenol and the primary amine until a more advanced stage and thus reduce the amount of free formaldehyde and free phenol, the dilutability of the resin, measured at 20° C., having to be maintained at least at 1000%.

After the P-F-A condensates have been formed, the reaction mixture is cooled so that its temperature reaches about 20 to 25° C. and is neutralized so as to stop the condensation reactions.

In general, the reaction mixture is neutralized by adding an acid in sufficient amount for the pH of the mixture to be less than 8.5, preferably less than 7.0 and advantageously between 4.0 and 6.0. The acid may be chosen from sulphuric, sulphamic, phosphoric and boric acids. Sulphuric acid and sulphamic acid are preferred.

The invention also relates to a sizing composition that can be applied to mineral fibres, especially glass or rock fibres, and to the insulating products obtained from these sized fibres.

The sizing composition comprises the phenolic resin according to the present invention and sizing additives.

Given that, as indicated above, the resin according to the invention has a very low free formaldehyde content of less than 0.3%, it is unnecessary to add urea to the sizing composition, except if it is desired to control the gel time of the size in order to prevent any pregelling problems.

In general, the sizing composition according to the invention comprises the following additives, per 100 parts of solid resin and urea material, where appropriate:

0 to 5 parts, generally less than 3 parts, of ammonium sulphate;

0 to 2 parts of a silane, in particular an aminosilane;

0 to 20 parts, generally 6 to 15 parts, of oil; and

0 to 20 parts, generally less than 12 parts, of aqueous ammonia (20 wt % solution).

The role of the additives is known and will be briefly recalled: the ammonium sulphate serves as a polycondensation catalyst (in the hot oven) after the sizing composition has been sprayed onto the fibres; the silane is a coupling agent for coupling between the fibres and the resin and also acts as an anti-ageing agent; the oils are hydrophobic anti-dust agents; aqueous ammonia acts, when cold, as a polycondensation retarder; and urea, as already mentioned, acts on the pregelling of the size.

The examples that follow allow the invention to be illustrated without however limiting it.

In the examples, the following analytical methods are used:

the amount of free phenol is measured by gas chromatography using a filled column (stationary phase: Carbowax 20 M) and a flame ionization detector (FID); and

the amount of free formaldehyde is measured by high-performance liquid chromatography (HPLC) and post-column reaction under the conditions of the ASTM D 5910-96 standard modified so that the mobile phase is water buffered to pH 6.8, the oven temperature is equal to 90° C. and the detection is carried out at 420 nm.

### EXAMPLE 1

Introduced into a two-litre reactor with a condenser on top and with a stirring system fitted, were 378 g of phenol (4 mol) and 809 g of formaldehyde (10 mol) as a 37% aqueous solution (formaldehyde/phenol molar ratio of 2.5) and the mixture was heated at 45° C. with stirring.

Next, 52.7 g of sodium hydroxide as a 50% aqueous solution (i.e. 7% by weight relative to the phenol) were regularly added over 30 minutes, the temperature was then progressively raised to 70° C. over 30 minutes, and this temperature was maintained for 80 minutes so as to reach a degree of phenol conversion of 93%.

Next, the temperature was reduced to 60° C. over 30 minutes and at the same time 75.3 g of monoethanolamine (1.2 mol) were introduced in a regular manner into the reaction mixture. The temperature was maintained at 60° C. for 15 minutes, a mixture was cooled down to about 25° C. over 30 minutes, and sulphamic acid as a 15% solution was added over 60 minutes until the pH was equal to 5.0.

The resin had the appearance of a clear aqueous composition having a water dilutability at 20° C. of greater than 1000% after 8 days of storage at 14° C. and after 21 days at 8° C.

The resin had a free formaldehyde content of 0.06% and a free phenol content of 0.2%, the content being expressed with respect to the total weight of liquid.

### EXAMPLE 2 (COMPARATIVE)

Preparation of a phenolic resin according to Example 4 of EP 0 480 778 A2 involving a secondary amine.

Introduced into the reactor of Example 1 were 564.66 g of phenol (6 mol) and 1217.43 g of formaldehyde (15 mol) as a 37% aqueous solution (formaldehyde/phenol molar ratio of 2.3) and the mixture was heated at 45° C. with stirring.

Next, 56.47 g of sodium hydroxide as a 50% aqueous solution (i.e. 5% by weight relative to the phenol) were regularly added over 30 minutes, then the temperature was progressively raised to 70° C. over 30 minutes, and the temperature maintained for 90 minutes so as to reach a degree of phenol conversion of 93%. Next, the temperature was reduced to 60° C. over 30 minutes and at the same time 124.22 g of diethanolamine (1.2 mol) were added regularly to the reaction mixture. The temperature was maintained at 60° C. for 15 minutes, the mixture was then cooled down to about 25° C. over 30 minutes, and sulphuric acid as a 25% solution was added over 60 minutes until the pH was equal to 8.0-8.1.

The resin had a free formaldehyde content of 1.0% and a free phenol content of 1.3%, the contents being expressed with respect to the total weight of liquid.

### EXAMPLE 3 (COMPARATIVE)

Preparation of a conventional urea-free phenolic resin.

Introduced into the reactor of Example 1 were 378 g of phenol (4 mol) and 857.7 g of formaldehyde (12.8 mol) as a 45% aqueous solution (formaldehyde/phenol molar ratio of 3.2) and the mixture was heated at 45° C. with stirring.

Next, 45.36 g of sodium hydroxide as a 50% aqueous solution (i.e. 6% by weight relative to the phenol) were regularly added, then the temperature was progressively raised to 70° C. over 30 minutes, and the temperature was maintained for 90 minutes so as to reach a degree of phenol conversion of 98%.

The mixture was cooled down to about 25° C. over 45 minutes and solid sulphamic acid was added over 60 minutes until the pH was equal to 7.3.

The resin had a water dilutability at 20° C. of greater than 1000% after 21 days of storage at 14° C.

The resin had a free formaldehyde content of 5.3% and a free phenol content of 0.41%, the contents being expressed with respect to the total weight of liquid.

1. A liquid resin, comprising phenol-formaldehyde and phenol-formaldehyde-amine condensates and having a water dilutability at 20° C. of at least 1000%, said liquid resin having a free formaldehyde content of 0.3% or less and a free phenol content of 0.5% or less, the contents expressed with respect to the total weight of liquid.

2. The resin according to claim 1, having a free formaldehyde content of 0.2% or less with respect to the total weight of liquid.

3. The resin according to claim 1, wherein the amine is a primary amine of formula:
R—NH₂
in which R represents a saturated or unsaturated, linear, branched or cyclic hydrocarbon group containing 1 to 10 carbon atoms.

4. The resin according to claim 3, wherein the hydrocarbon group R carries at least one hydroxyl radical.

5. The resin according to claim 4, wherein the amine is monoethanolamine.

6. The resin according to claim 1, having a free formaldehyde content of less than 0.2%, a free phenol content of less than 0.4% and a water dilutability of 2000% or higher.

7. A method of preparing a resin of comprising phenol-formaldehyde and phenol-formaldehyde-amine condensates and having a water dilutability at 20° C. of at least 1000%, a free formaldehyde content of 0.3% or less and a free phenol content of 0.5% or less, the contents expressed with respect to the total weight of liquid, in comprising
reacting the phenol and the formaldehyde in a formaldehyde/phenol molar ratio of greater than 1, in the presence of a basic catalyst,
cooling the reaction mixture and
introducing into said reaction mixture, during the cooling, an amine that reacts with the formaldehyde and the free phenol via the Mannich reaction, wherein a primary amine introduced right from the start of the cooling is used and in that the reaction mixture is maintained at the introduction temperature for a time varying from 10 to 120 minutes.

8. The method according to claim 7, wherein the duration of amine introduction varies from 20 to 100 minutes.

9. The method according to claim 7, wherein the formaldehyde reacts with the phenol in a formaldehyde/phenol molar ratio of between 2 and 4 and to a degree of phenol conversion of 93% or higher.

10. The method according to claim 7, wherein the introduction of the primary amine is performed at a temperature between 50 and 65° C.

11. The method according to claim 7, wherein the primary amine corresponds to the general formula:
R—NH₂
in which R represents a saturated or unsaturated, linear, branched or cyclic hydrocarbon group containing 1 to 10 carbon atoms.

12. The method according to claim 11, wherein the hydrocarbon group R carries at least one hydroxyl radial.

13. The method according to claim 12, wherein the amine is monoethanolamine.

14. The method according to claim 7, wherein the primary amine is added in an amount of 0.2 to 0.7 mol, per mole of starting phenol.

15. The method according to claim 7, wherein the reaction mixture, after cooling, is neutralized with an acid in an amount sufficient for the pH to be less than 8.5.

16. The method according to claim 7, wherein the acid is selected from the group consisting of sulphuric acid, sulphamic acid, phosphoric acid and boric acid.

17. A sizing composition for mineral fibres, comprising a phenolic resin according to claim 1 and sizing additives.

18. An insulation product, comprising mineral fibres sized with the sizing composition according to claim 17.

19. (canceled)

20. A liquid resin consisting essentially of phenol-formaldehyde and phenol-formaldehyde-amine condensates and having a water dilutability at 20° C. of at least 1000%, said liquid resin having a free formaldehyde content of 0.3% or less and a free phenol content of 0.5% or less, the contents expressed with respect to the total weight of liquid.

21. The resin according to claim 1, having a free formaldehyde content of 0.1% or less with respect to the total weight of liquid.

22. The resin according to claim 1, wherein the amine is a primary amine of formula:
R—NH₂
in which R represents a saturated or unsaturated, linear, branched or cyclic hydrocarbon group containing 1 to 6 carbon atoms.

23. A method of preparing a resin consisting essentially of phenol-formaldehyde and phenol-formaldehyde-amine condensates and having a water dilutability at 20° C. of at least 1000%, a free formaldehyde content of 0.3% or less and a free phenol content of 0.5% or less, the contents expressed with respect to the total weight of liquid, consisting of
reacting the phenol and the formaldehyde in a formaldehyde/phenol molar ratio of greater than 1, in the presence of a basic catalyst,
cooling the reaction mixture and
introducing into said reaction mixture, during the cooling, an amine that reacts with the formaldehyde and the free phenol via the Mannich reaction, wherein a primary amine introduced right from the start of the cooling is used and in that the reaction mixture is maintained at the introduction temperature for a time varying from 10 to 120 minutes.

24. The method according to claim 7, wherein the duration of amine introduction varies from 25 to 50 minutes.

25. The method according to claim 7, wherein the formaldehyde reacts with the phenol in a formaldehyde/phenol molar ratio of less than 3 and to a degree of phenol conversion of 93% or higher.

26. The method according to claim 7, wherein the introduction of the primary amine is performed at a temperature around 60° C.

27. The method according to claim 7, wherein the primary amine corresponds to the general formula:
R—NH₂
in which R represents a saturated or unsaturated, linear, branched or cyclic hydrocarbon group containing 1 to 6 carbon atoms.

28. The method according to claim 7, wherein the primary amine is added in an amount of 0.25 to 0.50 mol per mole of starting phenol.

29. The method according to claim 7, wherein the reaction mixture, after cooling, is neutralized with an acid in an amount sufficient for the pH to be less than 7.0.

30. The method according to claim 7, wherein the reaction mixture, after cooling, is neutralized with an acid in an amount sufficient for the pH to be between 4.0 and 6.0.

31. A sizing composition for mineral fibres, comprising a phenolic resin according to claim 1, urea and sizing additives.

32. A method of making the sizing according to claim 17, comprising mixing said phenolic resin and said sizing additives.

33. A method of making the insulation according to claim 18, comprising spraying said sizing composition onto said mineral fibres.
